(19) United States
(12) Patent Application Publication
(10) Pub. No.: US 2023/0338176 A1
(43) Pub. Date: Oct. 26, 2023

(54) Surgical method for endoscopic RYGBP ostomy revision

(71) Applicants: Allen Blosser, Arlington, VA (US); Carolina Sawley, Ft Lauderdale, FL (US)

(72) Inventors: Allen Blosser, Arlington, VA (US); Carolina Sawley, Ft Lauderdale, FL (US)

(21) Appl. No.: 17/860,565

(22) Filed: Jul. 8, 2022

Related U.S. Application Data
(60) Provisional application No. 63/333,403, filed on Apr. 21, 2022.

(51) Int. Cl. A61F 5/00; A61B 1/018; A61B 1/273; A61B 17/29

(52) U.S. Cl. CPC A61F 5/0086; A61B 1/018; A61B 1/2736; A61B 17/29; A61F 5/0089; A61B 17/1114

(57) ABSTRACT

A surgical method for restricting the size of a RYGBP patient's gastric outlet or “ostomy” by tightening an ostomy margin using existing in-market equipment to create banded tissue segments around the ostomy margin.

### CROSS REFERENCE TO RELATED APPLICATIONS

This patent application claims priority to U.S. provisional patent application 63/333,403, filed on Apr. 21, 2022.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

### Field of the Invention

The invention comprises a surgical method for restricting the size of a RYGBP patient's gastric outlet or “ostomy”, using existing in-market equipment as an alternative to TORe surgery, without the need for general anesthesia or an overnight hospital stay.

### Background of the Invention

Morbid obesity in the US is currently at pandemic scale with over 50% of the population obese or morbidly obese. Obese individuals that have previously undergone bariatric bypass surgery, specifically, Roux-en-Y Gastric Bypass (RYGBP) and have regained the excess weight originally lost have few options other to repeat the surgery, resulting in unnecessary health risks and financial inefficiency.

Transoral outlet reduction (TORe) is a minimally invasive, endoscopic revision procedure that can help RYGBP patients who have regained weight. While gastric bypass limits the amount of food a patient can eat and produces a feeling of fullness, its effectiveness can diminish over time. TORe tightens the gastric outlet or “ostomy” with sutures to decrease the size of the opening.

Known devices exist for suturing around the RYGBP ostomy in hopes of reducing the outlet orifice opening, thus restricting flow, and caloric intake, resulting in weight loss. Such known devices are difficult to manage, require general anesthesia and requires expert endoscopic skills to operate and must generally be performed on an inpatient basis. Currently less that 100 physicians in the U.S. are performing TORe with the Apollo device mainly due to the restrictive nature of the required apparatus and the skill level involved.

What is needed is an alternative post-RYGBP procedure that will tighten the patient's ostomy, which can be performed by most or all U.S. gastroenterologists and requires neither general anesthesia nor an overnight hospital stay.

### BRIEF SUMMARY OF THE INVENTION

In a preferred embodiment, a surgical procedure for restricting flow through the ostomy of a RYGBP patient comprising the following steps:
1. Sedating the patient with MAC anesthesia;
2. Performing a routine esophagogastroduodenoscopy (EGD) using an endoscope/gastroscope with a tube/channel;
3. Applying cautery circumferentially around an ostomy margin;
4. Passing an endoscopic tissue grasping device through the tube/channel, a distal end of such endoscopic tissue grasping device comprising forceps/graspers then grasping a tissue segment of the ostomy margin with a turning motion of said endoscopic tissue grasping device and retracting such device, thereby retracting the grasped tissue segment into the tube/channel of the endoscope/gastroscope;
5. Deploying a gastroscopic banding device, engaging the retracted tissue segment with a distal end of the gastroscopic banding device, then deploying a band to create a banded tissue area,
6. Retracting, disengaging and withdrawing the endoscopic grasping device;
7. Repeating steps 4 and 5 to create multiple banded tissue segments around the ostomy orifice; and
8. Withdrawing the endoscope, thus terminating the procedure.

In another preferred embodiment, the surgical procedure of as described herein, wherein the distal end of the grasping device comprises forceps/graspers embodied as a screw mechanism.

In another preferred embodiment, the surgical procedure of as described herein, wherein the turning motion of the tissue grasping device of step 4 comprises three turns and the unscrewing motion of step 5 also comprises three turns.

In another preferred embodiment, the surgical procedure of as described herein, wherein the endoscopic tissue grasping device grasps a tissue segment of 1-2 cm in length and pulls the tissue segment into the distal end of the banding device

In another preferred embodiment, the surgical procedure of as described herein, wherein the distal end of the gastroscopic banding device comprises a hollow piece that engages the tissue segment by overlaying the tissue segment.

In another preferred embodiment, the surgical procedure of as described herein, wherein the banding process of steps 4 and 5 is repeated four times, one for each quadrant around the ostomy orifice.

In another preferred embodiment, the surgical procedure of as described herein, further comprising step 9--Reintroducing endoscope to examine G-J anastomosis post therapy.

In another preferred embodiment, the surgical procedure of as described herein, wherein the cautery of step 3 is either BiCap or direct current cautery.

### BRIEF DESCRIPTION OF THE DRAWINGS

FIG. 1 is a line drawing evidencing deployment of an endoscope/gastroscope.

FIG. 2 is a line drawing evidencing the alimentary anatomy of a patient after Roux-en-Y Gastric Bypass surgery.

FIG. 3 is a line drawing evidencing a gastric ostomy after classic TORe surgery using sutures.

FIG. 4 is a line drawing evidencing a gastric ostomy after the inventive TORe-B surgical method using bands.

FIGS. 5A-5C are line drawings evidencing four examples of types of endoscopic tissue grasping devices that can be used in the inventive surgical method.

FIG. 6A-6B are line drawings evidencing the use of an endoscopic banding device to band tissue and 6C evidences multiple areas of banded tissue surrounding an ostomy.

FIG. 7 is a line drawing evidencing the application of a cautery around the ostomy margin.

FIG. 8A-8C are a line drawing evidencing grasping and banding of ostomy tissue.

### DETAILED DESCRIPTION OF THE INVENTION

Gastroscopy, also known as upper endoscopy, involves using a thin flexible tube (endoscope or gastroscope) to examine the upper digestive tract. One type of gastroscopy, Esophagogastroduodenoscopy (EGD), is an endoscopic procedure that allows the doctor to examine the patient's esophagus, stomach and duodenum. EGD is an outpatient procedure and takes only 30 to 60 minutes to perform. It is considered a minimally invasive procedure since it does not require an incision into one of the major body cavities and does not require any significant recovery after the procedure.

The invention is a surgical method for restricting the size of a RYGBP patient's gastric outlet or “ostomy” (hereinafter, “TORe-B”). The TORe-B application of existing in-market equipment offers an alternative to TORe, with equal or better results, and can be performed by any gastroenterologist using the techniques learned in fellowship training across the specialty. Additionally, TORe-B does not require general anesthesia and can be performed in routine outpatient GI endoscopic facilities.

In particular, the inventive procedure requires only monitored anesthesia care (MAC) sedation. MAC anesthesia is a type of sedation where the patient remains calm, aware of his surroundings and able to follow instructions as needed. It is typically administered through an IV into the skin and muscle around the area on which the surgery is performed.

The surgical instruments required for a TORe-B procedure include, without limitation, a gastroscope, a BiCap or direct current cautery device, an endoscopic tissue grasping device and a gastroscopic banding device.

In particular, tissue grasping instruments are well known in the field of endoscopic surgery. Such instruments are typically embodied as a set of at least two retractable forceps, sometimes called claws or jaws, which are typically hinged and attached to an elongated neck that can be extended through an endoscope to reach the surgical area inside a patient's alimentary tract. The opposite end of the instrument's elongated neck comprises one or more means of opening and closing the forceps, often embodied as handles, knobs, levers, slides, or similar mechanisms for manually opening and closing the forceps via one or more cords within the neck. Certain embodiments may contain an electronic means of manipulation such as a joystick that allows the doctor to reposition the forceps in addition to opening and closing them.

Gastroscopic banding instruments, sometimes referred to as ligation devices, are also known. These instruments deploy one or more bands, usually made of natural or synthetic rubber or a polymer with similar properties, to capture tissue within the patient's alimentary tract. Such instruments are often designed to carry multiple bands around a barrel-shaped distal end, with the barrel overlaying the tissue to be banded and a proximal end allowing the doctor to deploy the bands by pulling a wire or using a similar deployment mechanism. Such devices can thus ligate multiple tissue areas during a single deployment.

As employed in the inventive surgical procedure, a tissue grasping device is to be used in combination with a gastroscopic banding device to gather and band tissue around the margin of a patient's ostomy margin, thus gradually tightening the margin and decreasing the size of the ostomy after postoperative stretching of such tissue has occurred. The tissue is first secured with the grasping device, then ligated with the banding device. The tightening of one or more tissue areas on the ostomy margin allows the ostomy to again provide the passage-limiting function originally intended by the RYGBP procedure, thereby assisting the patient in continuing to maintain his or her decreased caloric intake and weight.

In practice, each of the grasping device and the banding device have a proximal end with user controls and a distal end for engaging tissue. Typically, the distal end of a grasping device will comprise a set of one or more forceps/graspers, while the distal end of a banding device will comprise a hollow piece, often barrel-shaped and surrounded by one or more bands, such hollow piece for holding a tissue segment and deploying a band around such tissue segment. Each device is extended through the endoscopic tube/channel. The distal end of the grasping device, or at least the forceps/graspers of the grasping device, emerge from the distal end of the endoscope's tube/channel to engage a segment of healthy tissue, gently pulling that tissue segment into the distal end of the banding device. Once the tissue segment is within the hollow piece of the banding device, the user deploys the band, and each device can be withdrawn. The length of a tissue segment to be grasped and banded ranges from 0.5 cm to 3 cm, preferably between 1-2 cm.

In a preferred embodiment, the grasping device holds the tissue until the band is deployed. In another iteration, the grasping device releases the tissue segment upon insertion into the banding device.

In a preferred embodiment, the grasping device pulls the engaged tissue segment into the tube/channel, where the banding device engages and bands the segment, which is then moved back outside by the grasping device before the grasping device is withdrawn. In another iteration, the distal ends of the grasping and banding device both engage the tissue entirely outside the endoscope tube/channel.

In particular, the inventive process is directed to the use of a single channel endoscope/gastroscope comprising an instrument channel with a 2.4-3.8 mm diameter, preferably a 2.8 mm diameter. Also preferred is the use of a tissue grasping device with forceps/graspers embodied as a screw mechanism incorporated into a catheter, to enable pulling of the grasped tissue into the endoscope/gastroscope channel, thus maximizing the amount of tissue to be banded and improving the efficacy of the tissue stretching effect on the ostomy margin.

In a primary embodiment, the TORe-B procedure involves the following steps:
1. Patient sedated by MAC anesthesia;
2. Routine esophagogastroduodenoscopy (EGD) performed;
3. Cautery applied (BiCap or direct current) circumferentially around ostomy margin;
4. Endoscopic tissue grasping device passed through working scope channel grasping tissue of the ostomy (preferably three turns) and retracted into the channel of a gastroscope;
5. A gastroscope banding device is deployed, capturing tissue, and grasping device unscrewed (preferably three turns) and withdrawn;
6. The banding process is repeated, placing multiple (preferably four) bands around the ostomy orifice; and
7. The gastroscope is withdrawn, thus terminating the procedure.

MAC anesthesia is considered safer than that of general anesthesia in that lower dosages of drugs are typically administered, with the dosages set for pain prevention in the patient's specific case, allowing for light, moderate or deep sedation. Unlike general anesthesia, no endotracheal tube is required for MAC. Further, MAC is associated with briefer hospital stays and lower 30-day mortality rates than general anesthesia.

BiCap cautery and direct current cautery are both types of electrocautery, wherein a metal wire electrode is delivered through an endoscope and activated, thus delivering current to cauterize tissue around the ostomy margin. The application of cautery at this point establishes an inflammatory process in the margin tissue to accelerate scar formation during the healing process.

The inventive procedure is unique in a number of respects:
1. The use of bands on grasped tissue to restrict the ostomy circumference;
2. The simultaneous use of endoscopic banding device and endoscopic tissue grasping device;
3. Two separate types of equipment not previously associated;
4. The procedure is possible using MAC anesthesia only;
5. The ability to perform the surgery on an outpatient basis;
6. Immediate results with fewer potential complications that current equipment on market; and
7. The ability of all U.S. trained endoscopists to perform the TORe-B procedure based on skills universally taught through the fellowship program.

The references recited herein are incorporated herein in their entirety, particularly as they relate to teaching the level of ordinary skill in this art and for any disclosure necessary for the common understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable equivalents.

### DETAILED DESCRIPTION OF THE FIGURES

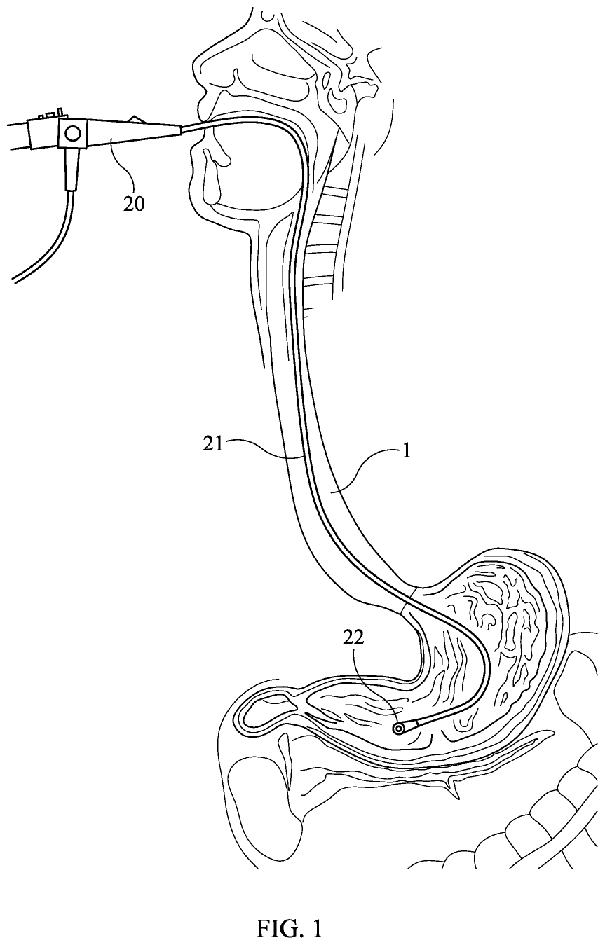

FIG. 1 is a line drawing evidencing standard deployment of an endoscope/gastroscope 20, with the endoscopic tube 21 extending down a patient's esophagus 1, terminating with a tube end 22.

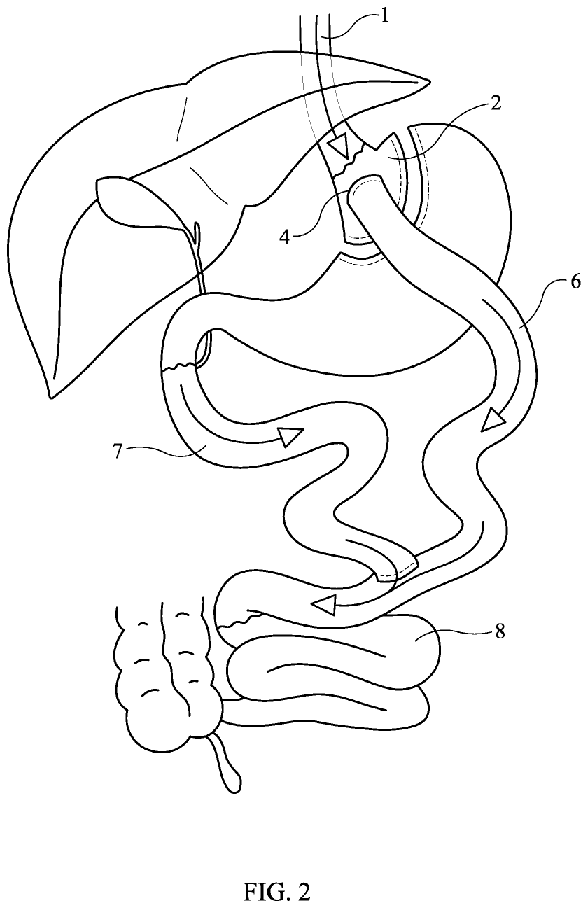

FIG. 2 shows the alimentary anatomy of a patient after Roux-en-Y Gastric Bypass surgery. In particular, the patient's stomach has been bifurcated with staples or a band, resulting in the esophagus 1 emptying into a small gastric pouch 2, now separated from the larger excluded stomach 3. The patient's intestine has been resected to create two pathways, first an ostomy 4 emptying from the small gastric pouch into a Roux limb 6 and thereby into the small intestine 8, and second a bilio-pancreatic limb emptying from the excluded stomach 3, also into the small intestine 8.

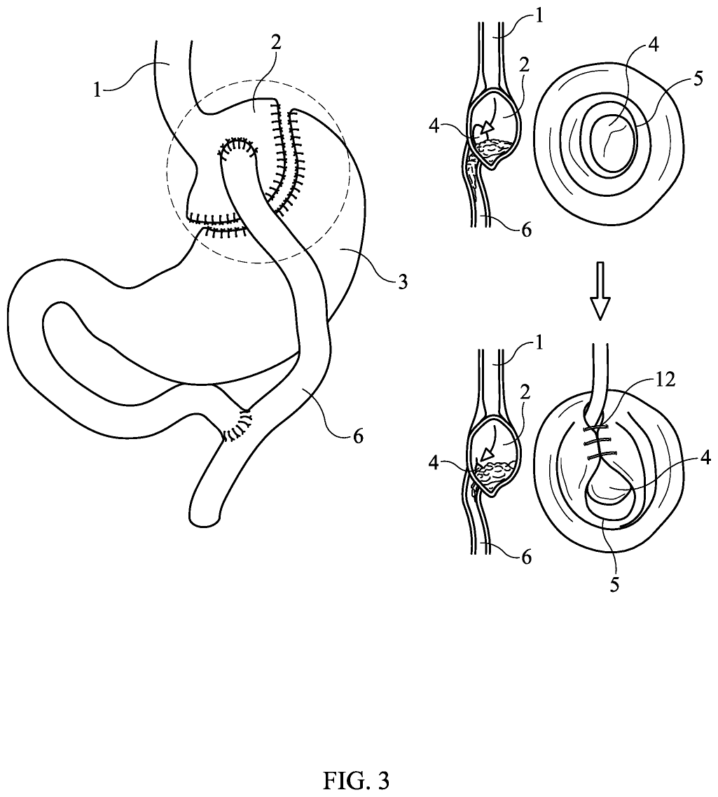

FIG. 3 shows multiple views of a gastric ostomy 4 after classic TORe surgery, wherein the gastric pouch 2 has been separated from the excluded stomach 3, the ostomy 4 empties into the Roux limb 6, and the ostomy margin 5 is partially closed to reduce throughflow with sutures 12.

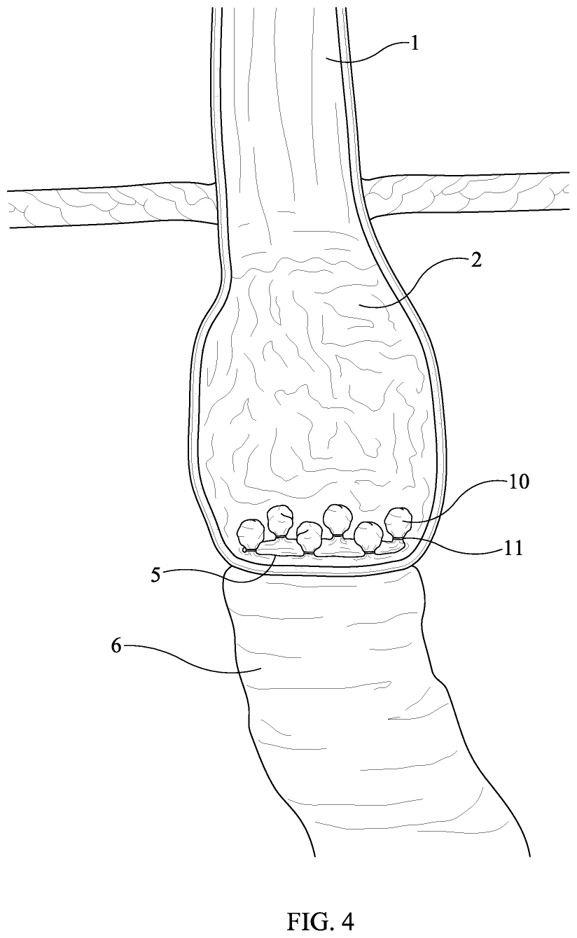

FIG. 4 shows a gastric ostomy margin 5 in a patient's gastric pouch 2 after the inventive TORe-B surgery wherein bands 11 have been used to create multiple banded tissue areas 10 around the ostomy margin, thus reducing the size of the ostomy 4 and limiting throughflow into the Roux limb 6.

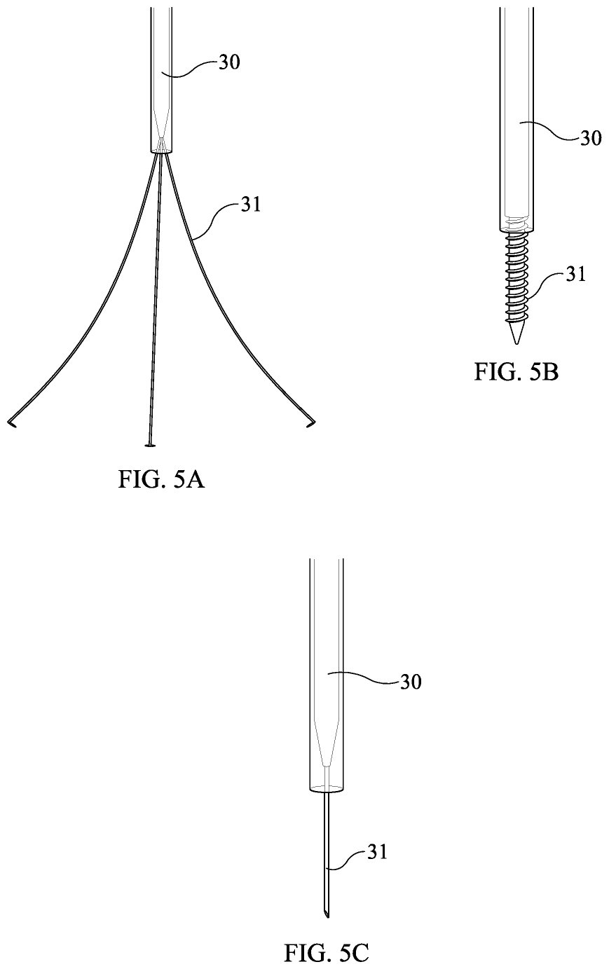

FIGS. 5A-5C shows known various iterations of an endoscopic tissue grasping device 30. FIG. 4A shows a device with multiple wire, barbed graspers 31, FIG. 4B shows a device with a screw-style grasper 31 and FIG. 4C shows a device with a needle-style grasper.

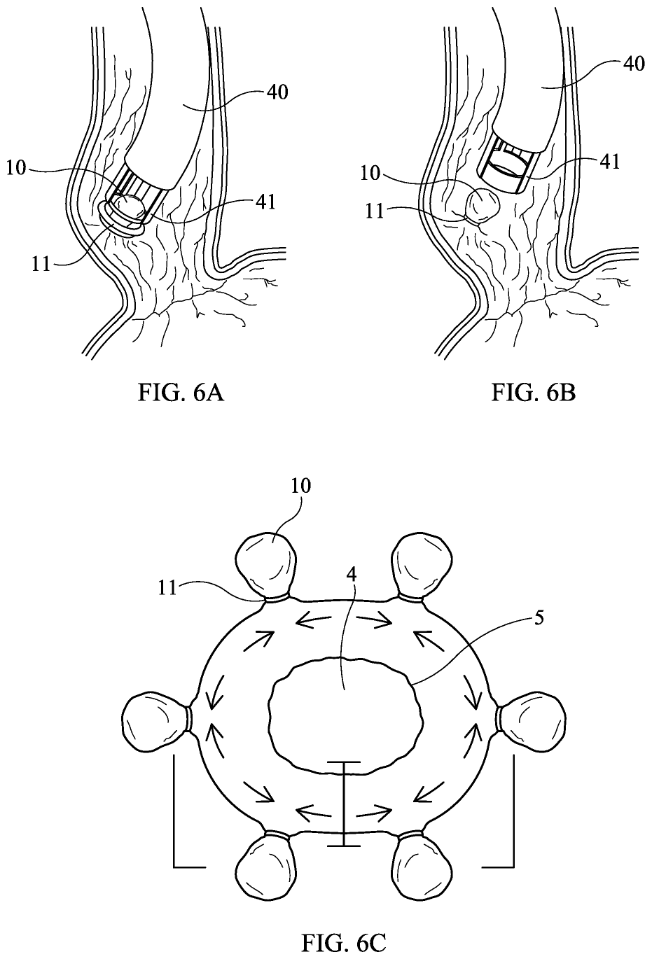

FIG. 6A shows a gastroscopic banding device 40 employed in holding and deploying a band 11 over an area of banded tissue 10, where the band 11 is sliding off of the device barrel 41. FIG. 6B shows the banded tissue area 10 after deployment of the band 11 as the device 40 is being retracted. FIG. 6C shows an ostomy 4 wherein a series of banded tissue areas 10, each with a band 11 at its base, surround the ostomy margin 5, thus exerting force across the margin tissue towards each banded tissue area and thus reducing the open portion of the ostomy.

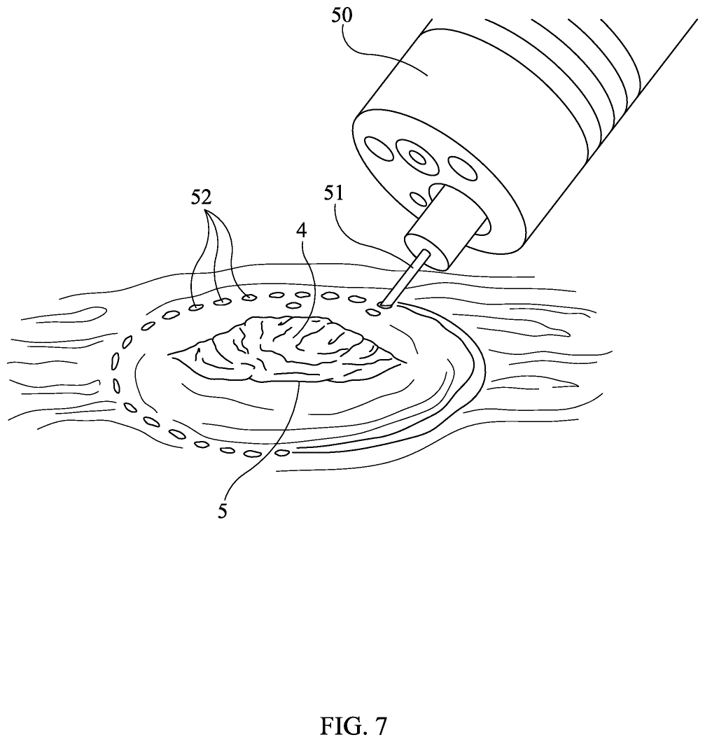

FIG. 7 shows a cautery instrument 50 deployed at an ostomy margin 5, applying heat to various cauterized tissue locations 52 around the margin to establish an inflammatory process in the margin tissue resulting in accelerated scar tissue formation during the healing process following the inventive procedure.

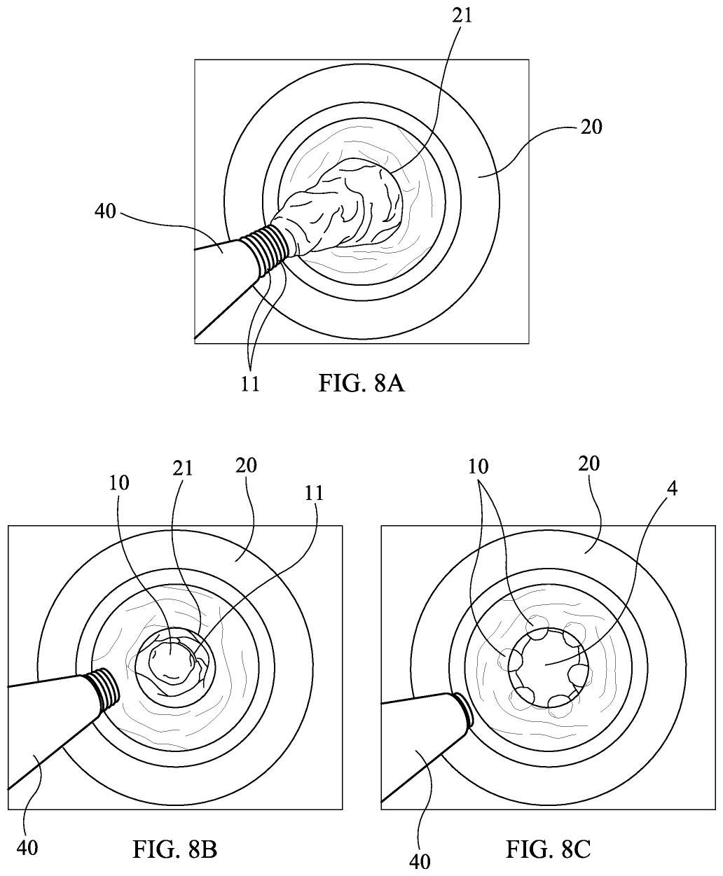

FIG. 8A-8C shows the use of a gastroscopic banding device 40. FIG. 8A shows the banding device 40 holding multiple bands 11 and pulling engaged tissue to be banded inside the tube 21 of an endoscope/gastroscope 20. FIG. 8B shows a newly-created area of banded tissue 10 surrounded by the band 11, again viewed through the tube 21 of the endoscope 20. Finally, FIG. 8C shows a series of areas of banded tissue 10 surrounding an ostomy 4 from within the tube 21 of the endoscope 20.

### PART NUMBERS

1 Esophagus
2 Gastric pouch
3 Excluded stomach
4 Ostomy
5 Ostomy margin
6 Roux limb
7 Bilio-pancreatic limb
8 Small intestine
9 Sutures
10 Banded tissue
11 Band
20 Endoscope/gastroscope
21 Tube
22 Tube end
30 Endoscopic tissue grasping device
31 Forceps/graspers
32 Doctor controls
40 Gastroscopic banding device
41 Barrel
50 Cautery instrument
51 Heated wire
52 Cauterized tissue

1. An endoscopic surgical procedure for restricting flow through the ostomy of a RYGBP patient comprising the following steps:
1. Sedating the patient with MAC anesthesia;
2. Performing a routine esophagogastroduodenoscopy (EGD) using an endoscope/gastroscope with a tube/channel;
3. Applying cautery circumferentially around an ostomy margin;
4. Passing an endoscopic tissue grasping device through the tube/channel, a distal end of such endoscopic tissue grasping device comprising forceps/graspers then grasping a tissue segment of the ostomy margin with a turning motion of said endoscopic tissue grasping device and retracting such device, thereby retracting the grasped tissue segment into the tube/channel of the endoscope/gastroscope;
5. Deploying a gastroscopic banding device, engaging the retracted tissue segment of the ostomy margin with a distal end of the gastroscopic banding device, then deploying a band to create a banded tissue area
6. Retracting, disengaging and withdrawing the endoscopic grasping device;
7. Repeating steps 4 and 5 to create multiple banded tissue segments around the ostomy orifice; and
8. Withdrawing the endoscope, thus terminating the procedure.

2. The endoscopic surgical procedure of claim 2, wherein the distal end of the grasping device comprises forceps/graspers embodied as a screw mechanism.

3. The endoscopic surgical procedure of claim 1, wherein the turning motion of the tissue grasping device of step 4 comprises three turns and the unscrewing motion of step 5 also comprises three turns.

4. The endoscopic surgical procedure of claim 1, wherein the endoscopic tissue grasping device grasps a tissue segment of 1-2 cm in length and pulls the tissue segment into the distal end of the banding device

5. The endoscopic surgical procedure of claim 1, wherein the distal end of the gastroscopic banding device comprises a hollow piece that engages the tissue segment by overlaying the tissue segment.

6. The endoscopic surgical procedure of claim 1, wherein the banding process of steps 4 and 5 is repeated four times, one for each quadrant around the ostomy orifice.

7. The endoscopic surgical procedure of claim 1, further comprising:

9. Reintroducing endoscope to examine G-J anastomosis post therapy.

8. The endoscopic surgical procedure of claim 1, wherein the cautery of step 3 is either BiCap or direct current cautery.
